(19) United States
(12) Patent Application Publication
(10) Pub. No.: US 2023/0255135 A1
(43) Pub. Date: Aug. 17, 2023

(54) SYSTEM AND METHOD FOR PRODUCING A FUEL PRODUCT

(71) Applicants: Compressed Energy Systems, LLC, Eldorado, IL (US); M&K Gas Company, LLC, Holdrege, NE (US)

(72) Inventor: Christopher E. SCHIMP, Eldorado, IL (US)

(21) Appl. No.: 17/671,792

(22) Filed: Feb. 15, 2022

(51) Int. Cl. A01C 3/02; C12M 1/107; C12M 1/00

(52) U.S. Cl. CPC A01C 3/023; C12M 21/04; C12M 45/04

(57) ABSTRACT

This disclosure relates to systems and methods for producing a fuel product from a source of organic matter which includes manure and may include other byproducts. The organic matter is processed by a teeter water feed hindered-bed separator device for substantially separating the animal manure from the other byproducts. The separated animal manure is then subjected to anaerobic digesting to produce the fuel product.

### TECHNICAL FIELD

The present disclosure is generally directed to the production of a fuel product, in particular systems and methods for processing livestock waste to obtain fuel and more particularly, systems and methods for creating fuel using anaerobic digesting.

### BACKGROUND

Many livestock farms, such as dairy farms and cattle ranches produce tremendous amounts of manure waste that must be properly managed. One form of management is to use manure waste to produce a source of fuel such as a methane biogas that can be converted to electricity or otherwise used as a source of fuel similar to natural gas.

In general, the invention described herein is a system and method for recovering manure in a livestock operation, such as a dairy or cattle operation. The manure may be recovered for purposes of supplying a anaerobic digester for extraction of energy from the biodegradable material.

Anaerobic digestion refers to a collection of processes by which organic matter is broken down by microorganisms in the absence of oxygen. Anaerobic digestion is used as part of a treatment process in the recycling of biodegradable waste matter such as foot waste, sewage sludge, or animal waste. The process results in the production of biogas, which is considered a source of renewal energy. Biogas can be further refined to produce bio methane, which has a similar methane content to natural gas.

There are several problems in anaerobic digestion of waste from beef cattle or dairy cows. Typically, the waste from such animals includes amounts of dirt from the feed lots or sand and other materials used in bedding for the livestock. The dirt, sand and other materials cause issues with the digesters such as clogging, which then requires significant down time to clean out. To date, there is no economical technology to remove the dirt, sand or other materials from the manure before it enters the anaerobic digesting process. This need is met by the systems and methods described herein, which feature the use of animal waste to form a fuel product.

### BRIEF DESCRIPTION OF DRAWINGS

To facilitate further description of the embodiments, the following drawings are provided, in which like references are intended to refer to like or corresponding parts, and in which:

FIG. 1 is a schematic view of a system for producing a fuel product from a source of organic matter;

FIG. 2 is a side view of an embodiment of a teeter water feed hindered-bed separator device that may be used as part of the system for producing a fuel product from a source of organic matter; and

FIG. 3 graphically represents a method for producing a fuel product from a source of organic matter.

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “top,” “bottom,” “upper,” “lower,” and the like in the description and in the claims, if any, are used for descriptive purposes and not necessarily for describing permanent relative positions. It is to be understood that the terms so used are interchangeable under appropriate circumstances such that the embodiments of the system and/or methods described herein are, for example, capable of operation in other orientations than those illustrated or otherwise described herein.

### DETAILED DESCRIPTION OF EXEMPLARY EMBODIMENTS

The present disclosure relates to a system and method for processing organic matter containing manure and other byproducts such as dirt, sand and other materials as part of a biogas digesting system in order to produce a fuel product. The method and system substantially separates and removes the manure from other byproducts or materials based on weight or density. The method and system uses the separated solids for feeding a digester that may be used to produce a fuel product such as a methane biogas.

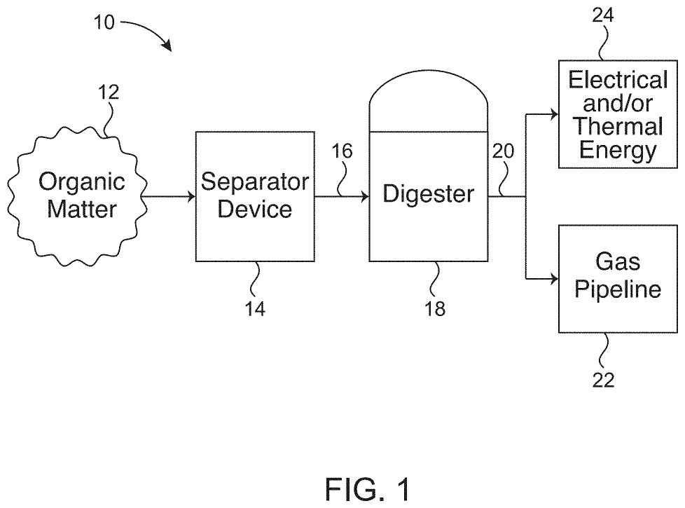

FIG. 1 is a schematic view of a system 10 for producing a fuel product from a source of organic matter 12, which may comprise animal manure and other byproducts such as dirt, sand and other materials. The organic matter 12 may be collected from that waste of livestock, such as beef cattle or dairy cows. The other materials or byproducts may be included in the animal waste due to the animals being housed in or fed in facilities or areas containing dirt or bedding that may include sand or other materials.

The organic matter 12 is fed into a separation device 14 such as a teeter water feed hindered-bed separator device. The separator device 14 substantially separates the animal manure 16 from the other materials and byproducts contained in the organic matter 12. The separated animal manure 16 may then be subjected to an anaerobic digester 18 to produce a biogas fuel product 20, which may then be processed as necessary for feeding into a natural gas pipeline 22 or used to generate electrical and/or thermal energy 24, among other uses.

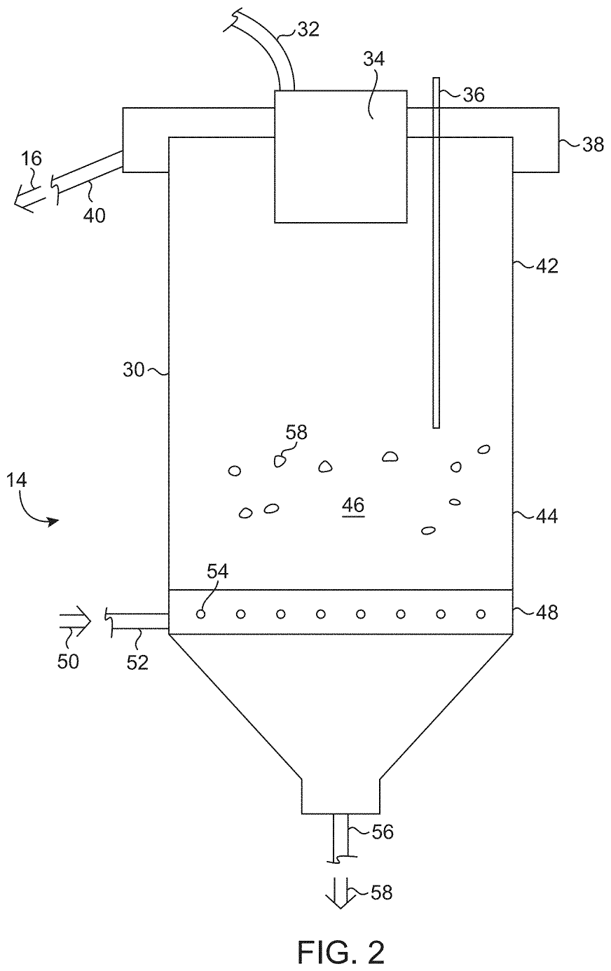

FIG. 2 depicts an embodiment of a teeter water feed hindered-bed separator device 14 that may be used as part of the system for producing a fuel product from a source of organic matter 12 consisting of animal manure and other byproducts. Although FIG. 2 depicts one form of a teeter water feed hindered-bed separator device, the present invention is not limited to any particular form of separator device.

As is known, a hindered-bed separator is a vessel in which water is evenly introduced across the base of the device and rises upward. The separator device 14 consists of a main housing 30 forming an enclosed chamber 32 that generally includes an upper portion 42 and a lower portion 44. A cylindrical feed well 34 is positioned centrally within the upper portion 42 and extends below an overflow launder 38. The feed well 36 is connected to an inlet feed line 32 into which the organic matter 12 is controllably fed.

The separator device 14 further includes an intake pipe 52 for receiving a source of teeter water inflow 50. The intake pipe 52 is connected to a series of water injection pipes to supply pressurized teeter water through a plurality of injection nozzles 54 preferably located evenly across a teeter plate 48. In such a manner, the teeter water 50 flows upwardly toward the feed well 34.

The slurry of organic matter 12 is introduced in the inlet 32 located in the upper end of the separator device 14 into the feed well 34. Teeter water 50 controllably enters the separator device through the intake pipe 52 and is evenly dispersed across the teeter plate 48 for dispersion through the injection nozzles 54.

The upward current of teeter water 50 creates a teeter zone 46 that suspends particles of a certain weight or density. Larger or more dense particles begin to settle downward against the flow of rising teeter water 50 at a rate defined by the particle size and/or density (specific gravity). The courser or denser solids settle at a rate that exceeds that of the rising water. These courser solids 58 may be discharged through the discharge outlet valve 56, which may be controlled by an electronic actuator that may be under computer control. The finer particles are maintained by the water flow in the upper portion 42 of the separator device 14 and discharged over the top and into the collection launder 38.

Rising water and light solids flow over the upper portion 42 of the separator device 14 and collect in an overflow launder 34. The light solids, which preferably consist of usable manure 16, may be discharged from separator device via outlet pipe 40.

The separator may also include a density control device 36 to measure the relative densities of the slurry in the upper and lower portions. If the measured density is greater than the desired density, the discharge outlet valve 56 may be regulated to adjust the average density of the teeter bed zone to a preselected standard value.

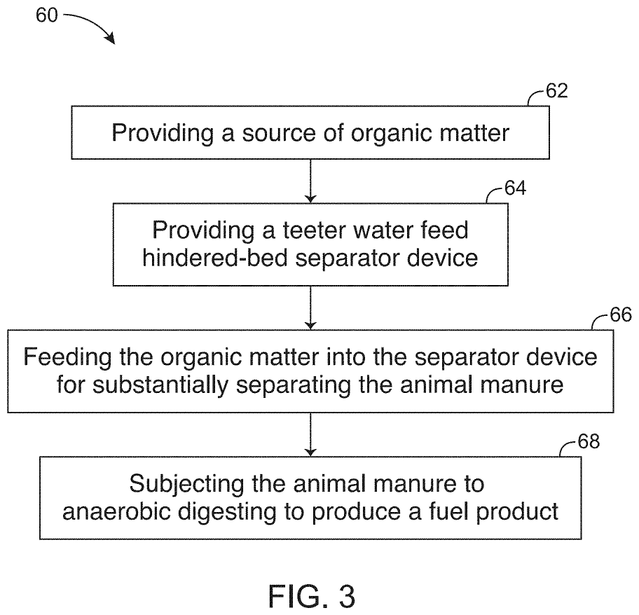

FIG. 3 is a method 60 for producing a fuel product. The method 60 begins at step 62 by providing a source of organic matter. Preferably, the organic matter includes livestock waste including animal manure, which may be contaminated with dirt, sand and other materials. The method continues at step 64 in which a teeter water feed hindered-bed separator device is provided. In step 66, the organic matter is fed into the separator device for substantially separating the animal manure from the other materials in the organic matter. In step 68, the separated animal manure is subjected to anaerobic digesting to produce a fuel product. The method 60 may then be repeated suing an additional source of organic matter.

While various novel features of the invention have been shown, described, and pointed out as applied to particular embodiments thereof, it should be understood that various omissions, substitutions and changes in the form and details of the systems and methods described and illustrated may be made by those skilled in the art without departing from the spirit of the invention. Amongst other things, the steps shown in the methods may be carried out in different orders in many cases, where such may be appropriate. Those skilled in the art will recognize, based on the above disclosure and an understanding therefrom of the teachings of the invention, that the particular hardware and devices that are part of the system described herein, and the general functionality provided by and incorporated therein, may vary in different embodiments of the invention. Accordingly, the particular system components are for illustrative purposes to facilitate a full and complete understanding and appreciation of the various aspects and functionality of particular embodiments of the invention, as realized in system and method embodiments thereof. Those skilled in the art will appreciate that the invention can be practiced in other than the described embodiments, which are presented for purposes of illustration and not limitation.

What is claimed is:

1. A method for producing a fuel product comprising the steps of:
providing a source of organic matter, the organic matter comprising animal manure and other byproducts;
providing a teeter water feed hindered-bed separator device;
feeding the organic matter into the separator device for substantially separating the animal manure from the other byproducts; and
subjecting the animal manure to anaerobic digesting to produce the fuel product.

2. The method of claim 1 wherein the separator device is a hindered-bed density separator device that separates the animal manure from the other byproducts based on the specific density of the manure.

3. The method of claim 1 wherein the fuel product is a methane biogas.

4. The method of claim 1 wherein the other byproducts include sand and/or dirt.

5. A system for producing a fuel product comprising:
a source of organic matter, the organic matter comprising animal manure and other byproducts;
a teeter water feed hindered-bed separator device for accepting the source of organic matter and separating the animal manure from the other materials; and
an anaerobic digester for producing the fuel product from the animal manure.

6. The system of claim 5, wherein the separator device comprises a hindered-bed separator.

7. The system of claim 6 wherein the hindered-bed separator comprises:
a main housing having an upper portion and a lower portion;
a feed inlet for feeding organic matter into the upper portion of the separator;
an inlet feeding teeter water that flows through a dispersing teeter plate and upward toward the upper portion of the main housing creating a zone of separation between the animal manure and the other materials;
an overflow launder collecting the separated animal manure; and
an outlet pipe for discharging the animal manure.

8. The system of claim 7 wherein the zone of separation may be adjusted for the specific gravity of the animal manure.
